(19) United States
(12) Patent Application Publication
(10) Pub. No.: US 2026/0226520 A1
(43) Pub. Date: Aug. 6, 2026

(54) PREPARATION METHOD AND APPLICATION OF A CHIRAL AMINE COMPOUND AND ITS SALTS

(71) Applicant: TONGLI BIOMEDICAL CO., LTD., Jiangsu (CN)

(72) Inventors: Mingxin QIAN, Jiangsu (CN); Donald C. HOU, Newtown, PA (US)

(21) Appl. No.: 19/646,151

(22) Filed: Apr. 13, 2026

Related U.S. Application Data
(63) Continuation-in-part of application No. PCT/CN2024/124708, filed on Oct. 14, 2024.
(60) Provisional application No. 63/590,003, filed on Oct. 13, 2023.

(51) Int. Cl. C12P 17/12; C12P 17/18

(52) U.S. Cl. CPC C12P 17/12; C12P 17/182

(57) ABSTRACT

The present application relates to a preparation method and application of a chiral amine compound and its salt. The method comprises the following steps: (1) causing compound 1 to react in the presence of imine reductase, NADP, glucose dehydrogenase, glucose, a solvent, and a co-solvent to obtain compound 2 and/or compound 2-A; (2) converting the compound 2 and/or compound 2-A into the chiral amine compound TL-010-2 and its salts.

### CROSS REFERENCE TO RELATED APPLICATIONS

This application claims priority to, and is a continuation-in-part of PCT Application No. PCT/CN2024/124708 filed Oct. 14, 2024, which claims priority to and the benefit of U.S. Application No. 63/590,003, filed on Oct. 13, 2023. The entire contents of each of the above applications are herein incorporated by reference.

### FIELD OF THE INVENTION

The present application relates to a preparation method and application of a chiral amine compound and its salt.

### BACKGROUND OF THE INVENTION

Previously we had developed and demonstrated a number of manufacturing routes for the production of (R)-praziquantel (levo-prazinquantel) starting from the key chiral intermediate, CP-5. Of note is that we had discovered a novel manufacturing process for the production of CP-5 using a lipase enzyme. Subsequent elaboration by several routes to the key intermediate, TL-010-02, or a protected version such as the N-Boc protected (a potential proposed RSM) was then achieved, which was then further processed to afford high quality (R)-praziquantel in several steps.

The manufacturing process to obtain high purity CP-5 is shown in Scheme 1 and is covered in U.S. Pat. No. 9,139,859 B2_Sep. 22, 2015 and WO2021/047566 A1_Mar. 18, 2021.

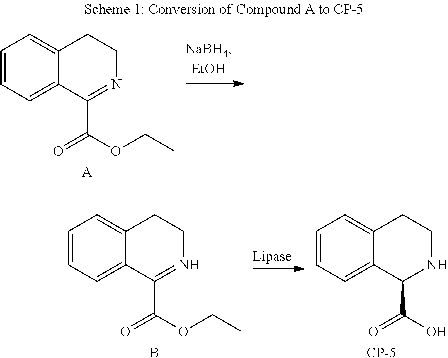

Conversion of CP-5 to the key intermediate TL-010-02 and subsequently to (R)-praziquantel was done by several approaches.

Numerous approaches have been conceived for the manufacture of (R)-praziquantel and one promising approach is the asymmetric hydrogenation of an appropriate substance to introduce the desired (R)-chirality. This has been reported by Czarnocki and co-workers (Roszkowshki, P.; Maurin, J. K.; Czarnocki, Z. Tetrahedron, Asymmetry, 2006, 17, 1415) for a substrate derived from phenylethylamine (See Scheme 2). However, the asymmetric transfer hydrogenation of imine Compound 4 to form Compound 5 is low yielding albeit with good enantioselectivity. The current recrystallization to upgrade the ee further lowers the overall yield to 24%. Thus, there is still room for further improvement.

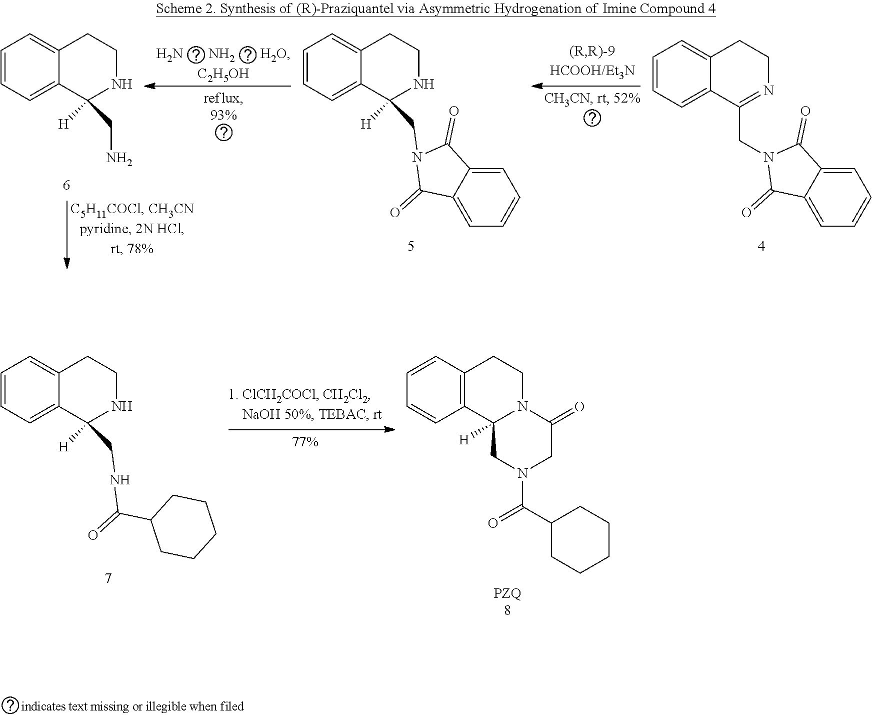 [SMILES: NC[C@@H]1NCCc2ccccc21 ; "H2N       NH2       H2O,C2H5OH" ; O=C1c2ccccc2C(=O)N1C[C@@H]1NCCc2ccccc21 ; O=C1c2ccccc2C(=O)N1CC1=NCCc2ccccc21 ; O=C(NC[C@@H]1NCCc2ccccc21)C1CCCCC1 ; O=C(C1CCCCC1)N1CC(=O)N2CCc3ccccc3[C@@H]2C1]

### SUMMARY OF THE INVENTION

One aspect of the present disclosure provides a method for preparing a chiral amine compound comprising the following steps:
(1) causing compound 1 to react in the presence of imine reductase, NADP, glucose dehydrogenase, glucose, a solvent, and a co-solvent to obtain compound 2 and/or compound 2-A;
(2) converting the compound 2 and/or compound 2-A into the chiral amine compound (1R)-(−)-1-aminomethyl-1,2,3,4-tetrahydroisoquinoline (TL-010-2);
wherein, the structural formula of the compound 1 is:

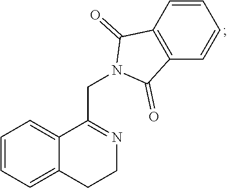 [SMILES: O=C1c2ccccc2C(=O)N1CC1=NCCc2ccccc21]

the structural formula of the compound 2 is:

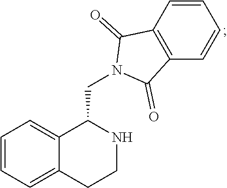 [SMILES: O=C1c2ccccc2C(=O)N1C[C@@H]1NCCc2ccccc21]

the structural formula of the compound 2-A is:

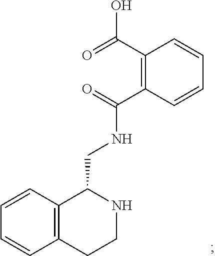 [SMILES: O=C(O)c1ccccc1C(=O)NC[C@@H]1NCCc2ccccc21]

the structural formula of the chiral amine compound TL-010-2 is

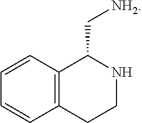 [SMILES: NC[C@@H]1NCCc2ccccc21]

The second aspect of the present disclosure is to provide a chiral amine di-hydrochloride salt having a structure of Formula

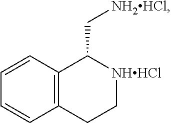

the di-hydrobromide salt having a structure of Formula

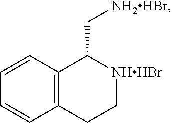

the oxalic acid salt having a structure of Formula

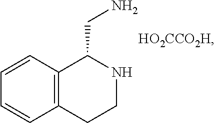 [SMILES: NC[C@@H]1NCCc2ccccc21]

and the maleic acid salt having a structure of Formula.

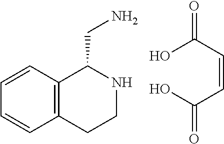 [SMILES: NC[C@@H]1NCCc2ccccc21 ; O=C(O)/C=C\C(=O)O]

The third aspect of the present disclosure is to provide a method for preparing (R)-praziquantel comprising the following steps:
1) causing compound 1 to react in the presence of imine reductase, NADP, glucose dehydrogenase, glucose, a solvent, and a co-solvent to obtain compound 2 and/or compound 2-A; 2) converting the compound 2 and/or compound 2-A into the key intermediate of (R)-praziquantel, TL-010-2; or further converting the chiral amine compound TL-010-2 into a salt of TL-010-2;
3) Preparing the (R)-praziquantel using the chiral amine compound TL-010-2 or the salt of TL-010-2 thereof as the starting material;
wherein, the structural formula of the compound 1 is:

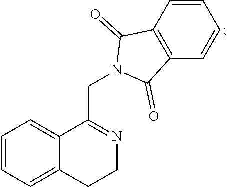 [SMILES: O=C1c2ccccc2C(=O)N1CC1=NCCc2ccccc21]

the structural formula of the compound 2 is

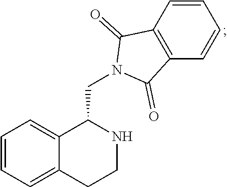 [SMILES: O=C1c2ccccc2C(=O)N1C[C@@H]1NCCc2ccccc21]

the structural formula of the compound 2-A is:

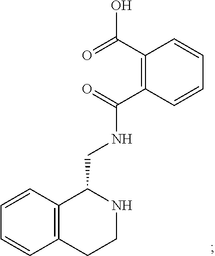 [SMILES: O=C(O)c1ccccc1C(=O)NC[C@@H]1NCCc2ccccc21]

the structural formula of the chiral amine compound TL-010-2 is

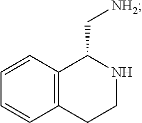 [SMILES: NC[C@@H]1NCCc2ccccc21]

and the structural formula of the salts of TL-010-2 are:

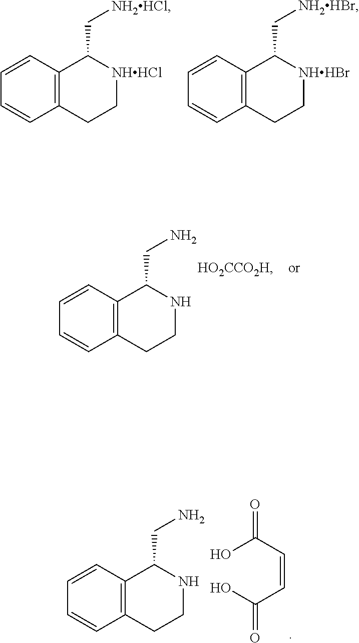

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a chromatogram showing the results of the HPLC for products compound 2 and 2-A.

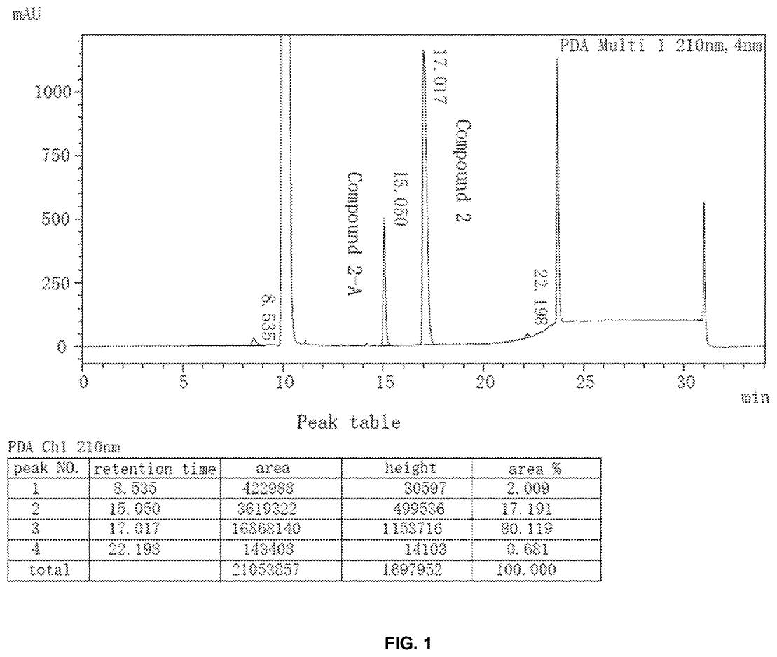

FIG. 2 is an NMR spectrum showing the results of ¹H-NMR for product compound 2.

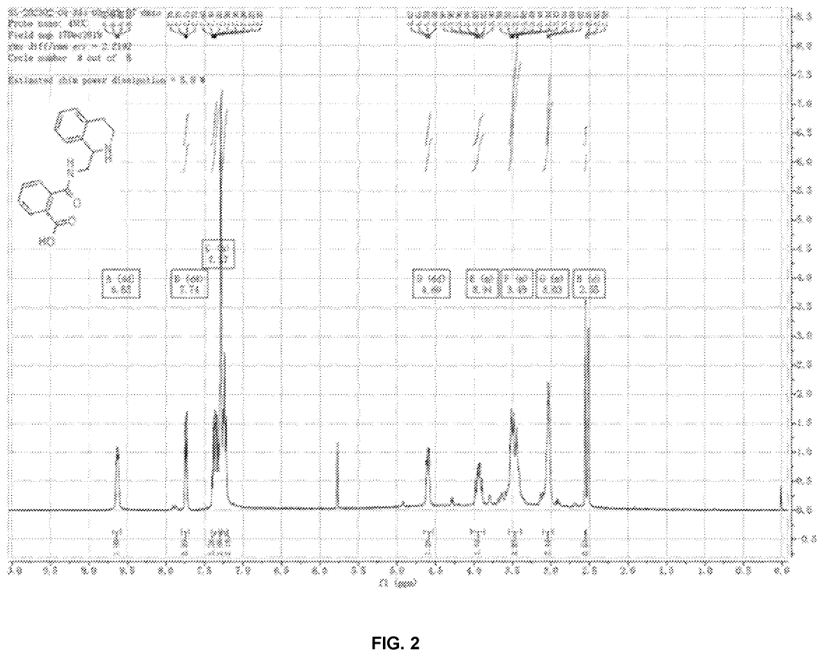

FIG. 3 is an NMR spectrum showing the results of ¹H-NMR for TL-010-2 free base.

FIG. 4 is a chromatogram showing the chiral HPLC chromatograph for TL-010-2 DiHCl salt.

FIG. 5 is an NMR spectrum showing the results of the 1H-NMR for TL-010-2 DiHCl salt.

FIG. 6 is an NMR spectrum showing the results of the 13C-NMR for TL-010-2 DiHCl salt.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Articles “a” and “an” are used herein to refer to one or more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including”, “comprising”, or “having”, and variations thereof, is meant to encompass the elements listed thereafter and equivalents thereof as well as additional elements. As used herein, “and/or” refers to and encompasses any and all possible combinations of one or more of the associated listed items, as well as the lack of combinations were interpreted in the alternative (“or”).

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10%~30%, or “1% to 3%”, etc., are expressly enumerated in this specification. These are only examples of what is specifically intended, and all possible combinations of numerical values between and including the lowest value and the highest value enumerated are to be considered to be expressly stated in this disclosure.

“ND” in this disclosure refers to the substance was not detected in the test.

Unless otherwise defined, all technical terms used herein have the same meaning as commonly understood by one of ordinary skilled in the art to which this disclosure belongs.

This application relates to an improved and innovative methods for preparing (R)-1-Aminomethyl-1,2,3,4-tetrahydroisoquinoline (TL-010-02) and salts and converting it to (R)-praziquantel.

The method comprises the following steps:

### Step 1.

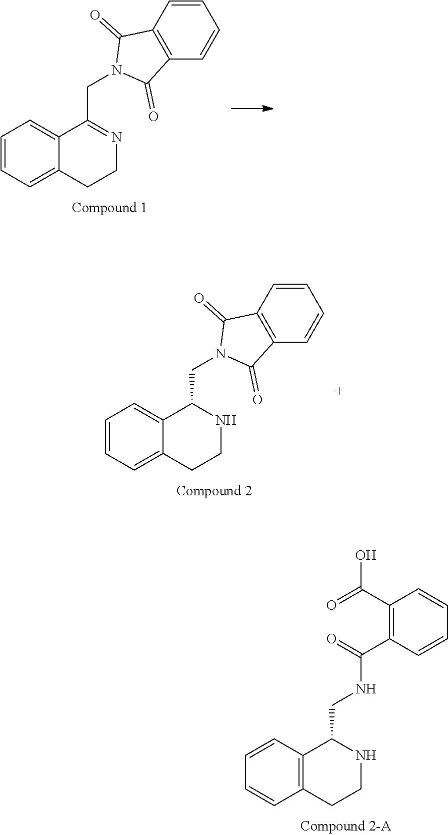 [SMILES: O=C1c2ccccc2C(=O)N1CC1=NCCc2ccccc21 ; O=C1c2ccccc2C(=O)N1C[C@@H]1NCCc2ccccc21 ; O=C(O)c1ccccc1C(=O)NC[C@@H]1NCCc2ccccc21]

By causing compound 1 to react in the presence of imine reductase, NADP, glucose dehydrogenase, glucose, a solvent, and a co-solvent, compound 2 and/or compound 2-A is obtained.

In some embodiments, the initial concentration of the compound 1 is 30 g/L to 100 g/L, such as 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L or 100 g/L. In some embodiments, the initial concentration of the compound 1 is 50 g/L to 100 g/L.

Initial concentration denotes the concentration of the substrate present in the system prior to the commencement of the reaction.

In some embodiments, in this step, the pH is maintained in the range of 5 to 7, such as 5, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. In some embodiments, in this step, the pH is maintained in the range of 6 to 6.5.

The pH can be maintained by adding alkaline solutions, including but not limited to sodium carbonate, sodium bicarbonate, potassium carbonate, calcium carbonate, sodium sulfite, sodium acetate, sodium sulfide, ferrous sulfide, sodium silicate, sodium phosphate, etc.

In some embodiments, in in this step, the reaction temperature is controlled to be 25° C. to 35° C., such as 25° C., 26° C., 27° C., 28° C., 29° C., 30° C., 31° C., 32° C., 33° C., 34° C., or 35° C. In some embodiments, in this step, the reaction temperature is controlled to be 25° C. to 30° C.

In some embodiments, the amount of NADP is 0.07 wt % to 1.5 wt % of the amount of compound 1, for example, the amount of NADP is 0.07 wt %, 0.1 wt %, 0.15 wt %, 0.2 wt %, 0.25 wt %, 0.3 wt %, 0.35 wt %, 0.4 wt %, 0.45 wt %, 0.5 wt %, 0.55 wt %, 0.6 wt %, 0.65 wt %, 0.7 wt % 0.75 wt %, 0.8 wt %, 0.85 wt %, 0.9 wt %, 0.95 wt %, 1 wt %, 1.05 wt %, 1.1 wt %, 1.15 wt %, 1.2 wt %, 1.25 wt %, 1.3 wt %, 1.35 wt %, 1.4 wt %, 1.45 wt % or 1.5 wt % of the amount of compound 1. In some embodiments, the amount of NADP is 0.1 wt % to 1.5 wt % of the amount of compound 1

In some embodiments, the molar amount of glucose is 1 to 3 times the molar amount of compound 1, for example, the molar amount of glucose is 1, 1.5, 2, 2.5 or 3 times the molar amount of compound 1. In some embodiments, the molar amount of glucose is 2 to 3 times the molar amount of compound 1.

In some embodiments, the amount of glucose dehydrogenase is 25 wt % to 75 wt % of the amount of compound 1, for example, the amount of glucose dehydrogenase is 25 wt %, 30 wt %, 35 wt %, 40 wt %, 45 wt %, 50 wt %, 55 wt %, 60 wt %, 65 wt %, 70 wt % or 75 wt % of the amount of compound 1. In some embodiments, the amount of glucose dehydrogenase is 40 wt % to 60 wt % of the amount of compound 1. Further, the amount of glucose dehydrogenase is 50 wt % of the amount of compound 1.

The amounts of the materials used in this application are calculated based on the ratio of the materials' addition amounts.

In some embodiments, the solvent is PBS buffer solution or water.

In some embodiments, the co-solvent is one or more of ethyl acetate, methanol, isopropyl acetate, dimethyl sulfoxide, toluene, methyl tert-butyl ether, isopropyl ether. Furthermore, the co-solvent is toluene.

In some embodiments, the amount of co-solvent used is sufficient to facilitate the reaction and can be added according to conventional amounts in the field. In some specific embodiments, the volume of co-solvent added is more than 10% of the total volume of the reaction materials. In addition, the volume of the co-solvent added is more than 20% of the total volume of the reaction materials, such as 20%, 22%, 24%, 26%, 28%, 30%, 32%, 34%, 36%, 38%, 40%, 42%, 44%, 46%, 48%, 50% and so on. In addition, the volume of the co-solvent added is between 20% and 40% of the total volume of the reaction materials. More specifically, the volume of co-solvent added is between 25% and 40% of the total volume of the reaction materials. More specifically, the volume of co-solvent added is 30% of the total volume of the reaction materials.

In some embodiments, the imine reductase is from Escherichia coli or Pichia pastoris, and fed in the form of enzyme powder, whole cells, cell disrupted fluid, or supernatant obtained from cell disrupted fluid after separation.

The cells, cell disrupted fluid, or supernatants obtained from cell disrupted fluid can be cells of Escherichia coli or Pichia pastoris that commonly express imine reductase.

In some embodiments, when the imine reductase is fed in the form of enzyme powder, the feeding quality of the imine reductase enzyme powder is 0.3 to 5 times the feeding quality of the compound 1, further 0.5 to 5 times a feeding quality of the compound 1, for example, the feeding quality of the imine reductase enzyme powder is 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 times the feeding quality of the compound 1. Further, the feeding quality of the imine reductase enzyme powder is 1 to 5 times the feeding quality of the compound 1. More further, the feeding quality of the imine reductase enzyme powder is 1.5 to 5 times the feeding quality of the compound 1. More further, the feeding quality of the imine reductase enzyme powder is 1.5 to 3 times the feeding quality of the compound 1.

In some embodiments, when the imine reductase is fed in the form of whole cells or cell disrupted fluid, the feeding quality of the whole cells or the feeding quality of the cells in the cell disrupted fluid is 1 to 10 times the feeding quality of the compound 1, for example, the feeding quality of the whole cells or the feeding quality of the cells in the cell disrupted fluid is 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6, 6.5, 7, 7.5, 8, 8.5, 9, 9.5 or 10 times the feeding quality of the compound 1. Further, the feeding quality of the whole cells or the feeding quality of the cells in the cell disrupted fluid is 3 to 10 times the feeding quality of the compound 1. More further, the feeding quality of the whole cells or the feeding quality of the cells in the cell disrupted fluid is 3 to 5 times the feeding quality of the compound 1.

In some embodiments, when the imine reductase is fed in the form of the supernatant obtained from cell disrupted fluid after separation, the feeding amount of the supernatant is 4 to 10 mL of the supernatant for every 1 g of the compound 1. For example, the feeding amount of the supernatant is 4 mL, 4.5 mL, 5 mL, 5.5 mL, 6 mL, 6.5 mL, 7 mL, 7.5 mL, 8 mL, 8.5 mL, 9 mL, 9.5 mL or 10 mL of the supernatant for every 1 g of the compound 1. Further, the feeding amount of the supernatant is 4 to 8 mL of the supernatant for every 1 g of the compound 1. More further, the feeding amount of the supernatant is 4 to 6 mL of the supernatant for every 1 g of the compound 1.

In some embodiments, the enzyme activity of the supernatant obtained from cell disrupted fluid after separation is 2~5 u/g.

In some embodiments, the supernatant obtained from cell disrupted fluid after separation is obtained by mixing, fragmenting and centrifuging whole cells expressing imine reductase with a solvent in a ratio of 1:3~6, wherein the solvent is PBS buffer or water.

The imine reductase and glucose dehydrogenase in this application can be enzymes of any type and from any source. The imine reductase and cells expressing it, glucose dehydrogenase, and other materials such as compound 1 in this application can be prepared using conventional methods in the field, or they can also be obtained commercially. For example, imine reductase can be obtained from TONGLI BIOMEDICAL CO., LTD. under the brand name TL-IRED, and glucose dehydrogenase can be obtained from TONGLI BIOMEDICAL CO., LTD. under the brand name TL-GDH, where the glucose dehydrogenase is added in the form of a crude enzyme solution. In the current disclosure, in general, the concentration of the glucose dehydrogenase crude enzyme solution is 50 g/L in the reaction system.

In some embodiments, after the completion of this step, a reaction mixture is subjected to centrifugation or vacuum distillation, DCM and/or methanol is added successively with stirring, and then filtered to collect an organic phase and a filter cake; a step of adding DCM and/or methanol to the filter cake, carrying out slurrying and filtration, and collecting an organic phase, which is performed once or multiple times; and the organic phases are combined and directly used for the reaction in next step.

### Step 2.

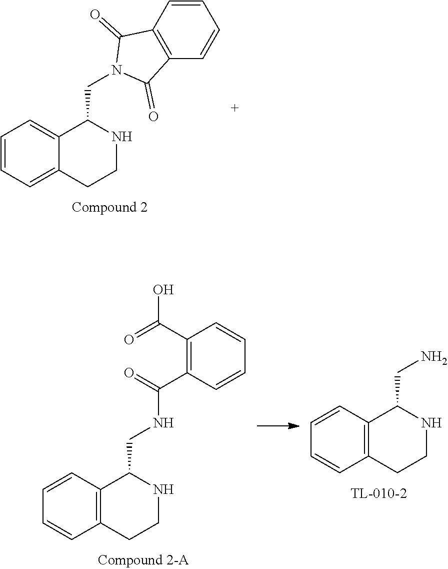 [SMILES: O=C1c2ccccc2C(=O)N1C[C@@H]1NCCc2ccccc21 ; O=C(O)c1ccccc1C(=O)NC[C@@H]1NCCc2ccccc21 ; NC[C@@H]1NCCc2ccccc21]

Compound 2 and/or compound 2-A are converted into the chiral amine compound, TL-010-2.

In some embodiments, the compound 2 and/or compound 2-A react in the presence of 85% hydrazine hydrate and methanol to obtain the chiral amine compound TL-010-2.

Research has found that both compound 2 and compound 2-A can be converted to TL-010-2 under the action of 85% hydrazine hydrate. Therefore, the organic phase after the post-treatment of Step 1 is used directly for deprotection using hydrazine hydrate without further purification.

According to some embodiments, the molar amount of 85% hydrazine hydrate is 2 to 4 times that of the molar amount of compound 1, further being 2.5 to 3.5 times.

The asymmetric reduction of compound 1 proceeds with high yield and high ee and is therefore significantly better than that reported by Czarnocki and co-workers.

STEP 3. The Step of Converting the TL-010-2 into its Salt

The salts of the chiral amine compound comprise hydrochloride, hydrobromide, oxalate, acetate, lactate, phosphate, maleate, tartrate, citrate, gluconate, sulfate, mesylate, or other sulfonate salts.

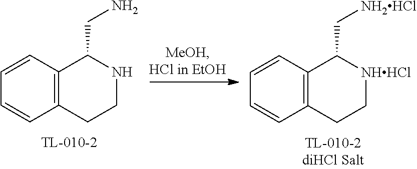

The chiral amine compound TL-010-2 is converted into TL-010-2 dihydrochloride salt in the presence of hydrochloric acid in EtOH and methanol.

In some embodiments, the specific steps for preparing the TL-010-2 dihydrochloride salt from the chiral amine compound TL-010-2 are as follows:
(1) Add the chiral amine compound TL-010-2 and methanol to a reactor and stir at room temperature;
(2) Under the state of being dissolved or undissolved, add an EtOH solution containing 20~25 wt. % HCl to the reactor while controlling the temperature at 15~30° C.;
(3) After the addition is complete, set the temperature to 75~85° C. and reflux and stir for 15~25 minutes;
(4) After the reaction is complete, the dihydrochloride salt of TL-010-2 is obtained through post-treatment.

This step can also be omitted, proceeding directly to step 4 with TL-010-2. If the intermediate needs to be stored for an extended period, it should be preserved in the form of the TL-010-2 dihydrochloride salt, and the dihydrochloride salt should be used as the raw material for the reaction in step 4.

The chiral amine compound TL-010-2 is converted into TL-010-2 dihydrobromide salt in the presence of hydrobromic acid and methanol, wherein the structural formula of TL-010-2 dihydrobromide salt is

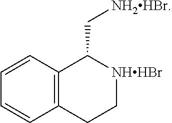

The chiral amine compound TL-010-2 is converted into TL-010-2 oxalate salt in the presence of oxalic acid and methanol, wherein the structural formula of TL-010-2 oxalate salt is

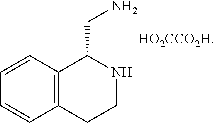 [SMILES: NC[C@@H]1NCCc2ccccc21]

The chiral amine compound TL-010-2 is converted into TL-010-2 maleic salt in the presence of maleic acid and methanol, wherein the structural formula of TL-010-2 maleic salt is

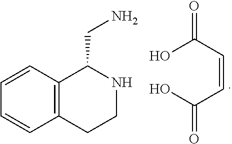 [SMILES: NC[C@@H]1NCCc2ccccc21 ; O=C(O)/C=C\C(=O)O]

### Step 4.

The chiral amine compound TL-010-2 or the salt of TL-010-2 is used as the starting material for preparing (R)-praziquantel.

The structural formula of the salts of TL-010-2 are:

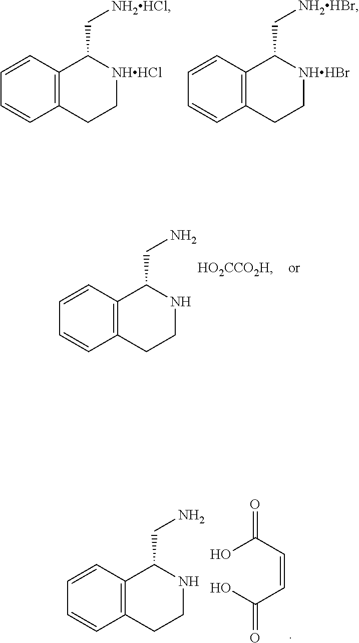

The specific reaction pathways and process parameters for preparing (R)-praziquantel are detailed in Reference (Roszkowshki, P.; Maurin, J. K.; Czarnocki, Z. Tetrahedron, Asymmetry, 2006, 17, 1415).

### Example 1: Screening Substrate Concentration

TABLE 1

| Reagent | Amount | |
|---|---|---|
| Compound 1 | 100.0 | mg |
| DMSO (Dimethyl sulfoxide) | 0.4 | mL |
| IRED (Imine Reductase) enzyme powder | 20 | mg |
| NADP | 0.7 | mg |
| GDH (Glucose dehydrogenase) | 50 | μL |
| glucose | 80.7 | mg |
| PBS buffer solution (pH: 5.6) | 2.7/1.4/0.4 | mL |

### Procedure:

1) The materials listed in Table 1 were added into a 10 mL single-necked flask and stirred, heated to a temperature of 30° C. for reaction. There were 3 experimental groups, due to different substrate concentration (about 30 g/L, 50 g/L, 100 g/L);
2) The sample was taken for HPLC analysis after a 22-hour reaction period;
3) The sample was taken for HPLC analysis after a 44-hour reaction period;
4) The sample was taken for HPLC analysis after a 70-hour reaction period;
5) ACN (10 mL) was added into the solvent and stirred for 10 min. Filtered and the filtrate was evaporated under reduced pressure;
6) DCM (dichloromethane) (10 mL) and H₂O (20 mL) were added and stirred. After phase separation, the organic layer was dried with anhydrous sodium sulfate and evaporated under reduced pressure, followed by column chromatography.

The HPLC data for the intermediate sample and the data for the product compound 2 after column chromatography are presented in Table 2.

TABLE 2

| concentration | 22 h IPC HPLC | | | 44 h IPC HPLC | | | 70 h IPC HPLC | | |
|---|---|---|---|---|---|---|---|---|---|
| of | Compound | Compound | Compound | Compound | Compound | Compound | Compound | Compound | Compound |
| substrate | 1 | 2 | 2-A | 1 | 2 | 2-A | 1 | 2 | 2-A |
| 30 g/L | 42.6% | 7.5% | 49.6% | 31.9% | 11.9% | 54.8% | 28.4% | 17.0% | 52.8% |
| 50 g/L | 18.9% | 17.3% | 62.9% | 17.2% | 19.0% | 62.5% | 15.0% | 18.9% | 63.6% |
| 100 g/L | 99.3% | 0.03% | 0.2% | 97.7% | 0.2% | 0.5% | 97.1% | 0.2% | 0.6% |

Results: The reaction of 50 g/L substrate concentration is good relative to the other concentrations, and the LC-MS and 1H-NMR of the product are displayed as target structures after purified with column chromatography.

### Example 2: Preliminary pH Screening

TABLE 3

| Reagent | Amount | |
|---|---|---|
| Compound 1 | 100.0 | mg |
| DMSO | 0.4 | mL |
| IRED Enzyme powder | 30 | mg |
| NADP | 0.7 | mg |
| GDH | 50 | μL |
| glucose | 80.7 | mg |
| PBS buffer solution | 2.7 | mL |

### Procedure:

1) The materials listed in Table 3 were added into a 10 mL single-necked flask and stirred magnetically, heated to a temperature of 30° C. for reaction. There were 5 experimental groups, due to different pH of buffer solution; the substrate concentration was about 30 g/L;
2) The sample was taken for HPLC analysis after a 22-hour reaction period;
3) The sample was taken for HPLC analysis after a 46-hour reaction period;
4) The sample was taken for HPLC analysis after a 70-hour reaction period.

The HPLC data for the intermediate samples are presented in Table 4.

TABLE 4

| | 22 h IPC | | | 46 h IPC | | | 70 h IPC | | |
|---|---|---|---|---|---|---|---|---|---|
| | Compound | Compound | Compound | Compound | Compound | Compound | Compound | Compound | Compound |
| pH | 1 | 2 | 2-A | 1 | 2 | 2-A | 1 | 2 | 2-A |
| 5.0 | 59.3% | 17.4% | 22.3% | 48.9% | 19.4% | 29.9% | 64.2% | 20.0% | 13.9% |
| 6.0 | 17.4% | 19.8% | 61.6% | 22.5% | 14.7% | 61.5% | 25.7% | 33.0% | 39.3% |
| 7.0 | 25.0% | 14.8% | 59.1% | 26.6% | 10.6% | 61.3% | 33.9% | 27.5% | 36.7% |
| 8.0 | 97.8% | 0.4% | 0.6% | 97.2% | 0.3% | 0.6% | 97.7% | 0.2% | 0.2% |
| 8.9 | 98.3% | 0.06% | 0.03% | 94.9% | 0.2% | 0.03% | 97.7% | 0.1% | 0.07% |

Results: As the time increases, the product increases, the pH of the system tends to become more alkaline and the amount of conversion to substrate compound 2-A increases. This is a dynamic change, indicating the need for constant monitoring and adjustment during the reaction and the importance of controlling the pH between 5.0 and 7.0. Subsequent fine sieving of the pH is required.

### Example 3: Further pH Screening

TABLE 5

| Reagent | Amount | |
|---|---|---|
| Compound 1 | 400.0 | mg |
| DMSO | 1.6 | mL |
| IRED Enzyme powder | 120 | mg |
| NADP | 2.8 | mg |
| GDH | 200 | μL |
| glucose | 322.7 | mg |
| PBS buffer solution | 5.8 | mL |

### Procedure:

1) The materials listed in Table 5 were added into a 10 mL single-necked flask and stirred magnetically, heated to a temperature of 30° C. for reaction. There were 4 experimental groups, due to different pH of buffer solution; the substrate concentration was about 50 g/L;
2) The sample was taken for HPLC analysis after a 24-hour reaction period;
3) The four reactions were combined, DCM (20 mL) and water (20 mL) were added, stirred for 10 min, filtered (with a lot of DE (diatomite), the filtrate was separated, the organic phase was discarded, and the aqueous phase was evaporated under pressure at 50° C. . . .
4) DCM (30 mL) was added and stirred for 30 min, filtered and obtain white solid of compound 2 and compound 2-A. The LC-MS test with MS (+) mode shows a mass of 311.

The intermediate HPLC data and the analytical data for compound 2 and compound 2-A are presented in Table 6.

TABLE 6

24 h IPC

| pH | Compound 1 | Compound 2 | Compound 2-A |
|---|---|---|---|
| 5.5 | 37.4% | 13.0% | 48.1% |
| 6.0 | 11.9% | 25.0% | 61.8% |
| 6.5 | 17.8% | 12.3% | 68.4% |
| 7.0 | 26.9% | 12.0% | 59.6% |

Results: Optimal buffer solution pH: 6.0~6.5.

### Example 4: Preliminary Temperature Screening

The material input for this example was identical to that of Example 3, and the pH of the buffer solution was 6.0. The experiment was completed according to the method of Example 3 at different reaction temperatures. Samples were taken at 24 and 48 hours of reaction, and HPLC was used for IPC (in-process control). The experimental data are shown in Table 7 below.

TABLE 7

| | 24 h IPC | | | 48 h IPC | | |
|---|---|---|---|---|---|---|
| T | Compound | Compound | Compound | Compound | Compound | Compound |
| ° C. | 1 | 2 | 2-A | 1 | 2 | 2-A |
| 20 | 36.9% | 29.4% | 32.4% | 23.6% | 30.0% | 45.7% |
| 25 | 22.7% | 40.5% | 35.1% | 11.8% | 39.1% | 47.1% |
| 30 | 14.6% | 34.6% | 49.2% | 21.3% | 30.3% | 46.9% |
| 35 | 32.1% | 34.4% | 31.3% | 30.5% | 35.8% | 30.8% |
| 40 | 55.7% | 20.0% | 22.1% | 67.2% | 14.7% | 16.3% |
| 50 | 83.1% | 7.0% | 6.1% | 90.2% | 4.3% | 3.7% |

Results: Optimal temperature is 25~30° C.

### Example 5: Screening of IRED Dosage

In this example, apart from the different amounts of IRED enzyme powder (120 mg/200 mg/320 mg/400 mg/600 mg, corresponding to 0.3 w/w, 0.5 w/w, 0.8 w/w, 1.0 w/w, 1.5 w/w) and the amount of glucose being 646.4 mg, the other components in the material input table are the same as in Example 4.

### Procedure:

1) The materials were added into a 10 mL single-necked flask and stirred magnetically, heated to a temperature of 30° C. for reaction. There were 5 experimental groups, due to different amounts of IRED enzyme powder;
2) The sample was taken for HPLC analysis after a 24-hour reaction period;
3) The sample was taken for HPLC analysis after a 48-hour reaction period.

The HPLC data for the intermediate samples are presented in Table 8.

TABLE 8

| The amount | 24 h IPC | | | 48 h IPC | | |
|---|---|---|---|---|---|---|
| of enzyme | Compound | Compound | Compound | Compound | Compound | Compound |
| powder(w/w) | 1 | 2 | 2-A | 1 | 2 | 2-A |
| 0.3 | 8.6% | 17.1% | 72.6% | 14.5% | 17.6% | 66.0% |
| 0.5 | 10.8% | 21.2% | 65.8% | 12.9% | 21.6% | 63.0% |
| 0.8 | 10.7% | 24.1% | 62.1% | 13.9% | 22.9% | 59.9% |
| 1.0 | 7.9% | 28.6% | 60.4% | 7.0% | 28.9% | 60.3% |
| 1.5 | 3.5% | 32.0% | 59.7% | 2.5% | 33.2% | 59.5% |

Results show using enzyme powder at 0.3 w/w [symbol] 0.5 w/w [symbol] 0.8 w/w [symbol] 1.0 w/w has similar results. As the amount of enzyme increases, the substrate (compound 1) tends to react almost completely.

### Example 6: Optimizing the Amount of E. coli Cell Expressing Imine Reductase

TABLE 9

| Reagent | Amount | |
|---|---|---|
| Compound 1 | 400.0 | mg |
| DMSO | 1.6 | mL |
| IRED cell | 400 [symbol] 800 [symbol] 1200 [symbol] 1600 | mg |
| NADP | 2.8 | mg |
| GDH | 200 | μL |
| glucose | 646.4 | mg |
| PBS buffer | 5.2 [symbol] 4.8 [symbol] 4.4 [symbol] 4.0 | mL |
| solution (pH 6.5) | | |

### Procedure:

1) 4 10 mL single-necked flasks were taken, each material in Table 9 (concentration of substrate was about 50 g/L) was added to each 10 mL single-necked flask and stirred magnetically, and heated to 30° C.;
2) The sample was taken for HPLC analysis after a 24-hour reaction period;
3) The sample was taken for HPLC analysis after a 48-hour reaction period.

The HPLC data for the intermediate samples are presented in Table 10.

TABLE 10

| The amount | 24 h IPC | | | 48 h IPC | | |
|---|---|---|---|---|---|---|
| of cell | Compound | Compound | Compound | Compound | Compound | Compound |
| (w/w) | 1 | 2 | 2-A | 1 | 2 | 2-A |
| 1 | 13.9% | 21.6% | 63.5% | 18.6% | 23.0% | 56.4% |
| 2 | 13.0% | 29.9% | 56.7% | 14.7% | 29.3% | 54.2% |
| 3 | 7.9% | 33.5% | 58.0% | 8.2% | 34.3% | 55.6% |
| 4 | 2.5% | 40.6% | 56.6% | 1.9% | 34.2% | 61.8% |

Results show that with amount of cell at 4 w/w, the starting material was almost fully converted to the products.

### Example 7: Optimizing the Amount of E. coli Cell Disrupted Fluid Expressing Imine Reductase

TABLE 11

| Reagent | Amount | |
|---|---|---|
| Compound 1 | 400.0 | mg |
| DMSO | 1.6 | mL |
| IRED cell disrupted fluid | 2000 [symbol] 4000 [symbol] 6000 [symbol] 8000 | mg |
| (20% content of cell) | | |
| NADP | 2.8 | mg |
| GDH | 200 | μL |
| glucose | 646.4 | mg |
| PBS buffer solution | 3.6 [symbol] 1.6 [symbol] 0 [symbol] 0 | mL |

Wherein, IRED cell disrupted fluid (20% content of cell): the mixture obtained from the preparation of 5 g cells/20 mL PBS buffer solution (pH 6.5) was used to disrupt the cells by ultrasound and other means to obtain the cell disrupted fluid.

### Procedure:

1) The materials listed in Table 11 were added into a 10 mL single-necked flask and stirred magnetically, heated to a temperature of 30° C. for reaction;
2) The sample was taken for HPLC analysis after a 24-hour reaction period;
3) The sample was taken for HPLC analysis after a 48-hour reaction period.

The HPLC data for the intermediate samples are presented in Table 12.

TABLE 12

| | 24 h IPC | | | 48 h IPC | | |
|---|---|---|---|---|---|---|
| | Compound | Compound | Compound | Compound | Compound | Compound |
| The amount of cell (w/w) | 1 | 2 | 2-A | 1 | 2 | 2-A |
| 5 (amount of cell 1 w/w) | 11.5% | 29.6% | 57.8% | 18.1% | 31.0% | 49.1% |
| 10 (amount of cell 2 w/w) | 9.1% | 32.6% | 57.1% | 13.4% | 32.5% | 51.8% |
| 15 (amount of cell 3 w/w) | 7.9% | 31.6% | 58.8% | 10.0% | 27.8% | 59.3% |
| 20 (amount of cell 4 w/w) | 1.0% | 31.6% | 65.9% | 0.9% | 26.6% | 70.2% |

Results show that with cell disrupted fluid at 20 w/w, the raw material was almost fully converted. The results of the reaction between the cells and the cell disrupted fluid are similar.

### Example 8: Screening the Better Cosolvent

TABLE 13

| Reagent | Amount | |
|---|---|---|
| Compound 1 | 4.0 | g |
| Cosolvent | 8 | mL |
| IRED cell | 4.0 | g |
| NADP | 28 | mg |
| GDH | 2 | mL |
| glucose | 6.5 | g |
| 0.1M PBS buffer solution (pH 6.5) | 55.5 | mL |

### Procedure:

1) The materials listed in Table 13 (concentration of substrate was about 50 g/L) were added into a 100 mL single-necked flask and stirred magnetically, heated to 27° C. for reaction (11 groups);
2) The initial pH was 6.3-6.5. The pH was adjusted to 6.3 by adding 1N HCl. The pH was reduced during the reaction, and the pH was stabilized at 6.3 by the addition of a 20% sodium carbonate solution;
3) Two samples were taken for HPLC analysis after a 24-hour reaction period;
4) Two samples were taken for HPLC analysis after a 48-hour reaction period;

In the reaction using toluene or isopropyl ether as a solvent, the mixture was concentrated under reduced pressure at 50° C., then a paste was made with methanol (MeOH), followed by filtration. The MeOH phase was tested for chirality.

The data for the samples are presented in Table 14.

TABLE 14

| | 24 h IPC | | | 48 h IPC | | |
|---|---|---|---|---|---|---|
| | Compound | Compound | Compound | Compound | Compound | Compound |
| cosolvent | 1 | 2 | 2-A | 1 | 2 | 2-A |
| DMSO | 18.3% | 8.1% | 72.1% | 18.1% | 29.7% | 50.4% |
| ACN | 94.0% | 0.5% | 2.9% | 91.0% | 2.4% | 2.8% |
| THF | 98.3% | ND | 0.03% | 96.1% | ND | 0.02% |
| MeOH | 41.5% | 11.6% | 43.2% | 28.5% | 20.9% | 46.4% |
| EtOH | 61.6% | 7.1% | 26.9% | 58.4% | 18.1% | 18.6% |
| IPA | 92.3% | 0.7% | 1.8% | 92.6% | 1.3% | 1.1% |
| EA | 93.4% | 1.0% | 2.4% | 91.6% | 1.7% | 2.5% |
| MTBE | 19.8% | 44.7% | 33.3% | 18.3% | 30.6% | 48.2% |
| Toluene | 27.6% | 43.0% | 26.9% | 21.9% | 39.6% | 31.9% |
| Isopropyl ether | 13.9% | 46.2% | 37.7% | 11.8% | 45.1% | 40.8% |
| Iso-BuOH | 96.5% | 0.02% | 0.03% | 94.7% | 0.01% | 0.02% |

In Table 14 above, the preliminary screening results indicate that the cosolvents DMSO, MTBE, isopropyl ether, and toluene are better. Further amplification and comparison experiments found that adding water-soluble cosolvents will worsen the reaction, and the effect of some cosolvents is low. Therefore, toluene is used as a cosolvent.

### Example 9: Rescreening for pH

TABLE 15

| Reagent | Amount | |
|---|---|---|
| Compound 1 | 4.0 | g |
| Toluene | 4 | mL |
| IRED cell | 4.0 | g |
| NADP | 28 | mg |
| GDH | 2 | mL |
| glucose | 6.5 | g |
| 0.1M PBS buffer solution | 19.5 | mL |

### Procedure:

1) The materials (concentration of substrate: 100 g/L) were added into a 100 mL single-necked flask and stirred magnetically, heated to 27° C. for reaction;
2) The pH was reduced during the reaction, and the pH was stabilized at 6.2, 6.3 or 6.4 by the addition of a 20% sodium carbonate solution;
3) The sample was taken for HPLC analysis after a 14-hour reaction period;
4) The sample was taken for HPLC analysis after a 37-hour reaction period.

The data for the samples are presented in Table 16.

TABLE 16

| | 14 h IPC | | | | 37 h IPC | | | |
|---|---|---|---|---|---|---|---|---|
| | Compound | Compound | Compound | | Compound | Compound | Compound | |
| pH | 1 | 2 | 2-A | ee | 1 | 2 | 2-A | ee |
| 6.2 | 15.8% | 54.8% | 27.2% | 99.4% | 7.2% | 72.2% | 18.7% | 99.7% |
| | 15.6% | 47.7% | 34.7% | | 7.4% | 65.6% | 24.8% | |
| 6.3 | ND | 85.7% | 11.9% | 99.2% | ND | 78.2% | 19.0% | 99.6% |
| | ND | 82.8% | 14.6% | | ND | 80.1% | 17.2% | |
| 6.4 | 2.7% | 83.2% | 12.0% | 99.3% | ND | 64.3% | 33.0% | 99.6% |
| | 3.0% | 80.4% | 13.9% | | ND | 58.4% | 38.2% | |

Results: Optimal pH is about 6.3.

### Example 10: Screening the Amount of NADP

TABLE 17

| | |
|---|---|
| Reagent | Amount |

| | | |
|---|---|---|
| Compound 1 | 20.0 | g |
| Toluene | 60.0 | mL |
| IRED cell | 20.0 | g |
| NADP | 280 [symbol] 210 [symbol] 140 [symbol] 70 [symbol] 14 | mg |
| GDH | 10.0 | mL |
| glucose | 32.3 | g |
| 0.1M PBS buffer solution | 57.7 | mL |

### Procedure:

1) The materials (concentration of substrate: 100 g/L) were added into a 250 mL single-necked flask and stirred mechanically, heated to 27° C. for reaction;
2) The pH was reduced during the reaction, and the pH was stabilized at 6.3 by the addition of a 20% sodium carbonate solution;
3) The sample was taken for HPLC analysis after a 24-hour reaction period.

The data for the samples are presented in Table 18.

TABLE 18

24 h IPC

| NADP | Compound 1 | Compound 2 | Compound 2-A |
|---|---|---|---|
| 1.4% | 0.5% | 91.7% | 4.1% |
| | 0.4% | 92.3 | 4.2% |
| 1.05% | 1.4% | 83.8% | 11.3% |
| | 1.0% | 83.5% | 12.1% |
| 0.7% | 3.8% | 77.9% | 15.4% |
| | 3.8% | 75.1% | 17.8% |
| 0.35% | 5.7% | 76.1% | 15.5% |
| | 5.9% | 74.1% | 17.0% |
| 0.07% | 17.6% | 64.2% | 15.7% |
| | 16.5% | 66.4% | 15.1% |

Results: As the amount of NADP increases, the reaction becomes faster and nearly complete.

### Example 11: The Equivalent of Glucose is 1.4 Eq-2.6 Eq

TABLE 19

| Reagent | Amount | |
|---|---|---|
| Compound 1 | 20.0 | g |
| Toluene | 20.0 | mL |
| IRED cell | 20.0 | g |
| NADP | 140.0 | mg |
| GDH | 10.0 | mL |
| glucose | 32.3 [symbol] 24.8 [symbol] 17.4 | g |
| 0.1M PBS buffer solution | 57.7 [symbol] 65.2 [symbol] 72.6 | mL |

### Procedure:

1) The materials (concentration of substrate was about 100 g/L) were added into a 250 mL three-necked flask with jacket and stirred mechanically, heated to 27° C. for reaction;
2) The pH was stabilized at 6.3 by the addition of a 20% sodium carbonate solution;
3) The sample was taken for HPLC analysis after a 24-hour reaction period.

The data for the samples are presented in Table 20.

TABLE 20

| | 24 hIPC | | |
|---|---|---|---|
| Glucose (eq) | Compound 1 | Compound 2 | Compound 2-A |
| 2.6 | 0.8% | 71.9% | 21.7% |
| | 0.8% | 74.6% | 19.7% |
| 2.0 | 1.6% | 80.5% | 14.0% |
| | 1.6% | 80.9% | 13.5% |
| 1.4 | 8.1% | 61.3% | 23.2% |
| | 7.1% | 66.7% | 20.3% |

Results: Optimal equivalent is 2.6 eq.

### Example 12: Feeding the Supernatant of the Lysate of E. coli Cells Expressing Imine Reductase

TABLE 21

| Reagent | Amount | |
|---|---|---|
| Compound 1 | 100.0 | g |
| Toluene | 300.0 | mL |
| IRED enzyme supernatant | 400.0 | mL |
| NADP | 350.0 | mg |
| GDH | 50.0 | mL |
| glucose | 161.3 | g |

The IRED enzyme supernatant is the supernatant obtained from the cell disrupted fluid after centrifugation or sedimentation. The enzyme activity of the supernatant separated from the cell disrupted fluid is 3.7 u/g.

### Procedure:

1) The materials (concentration of substrate: 100 g/L) were added into a 3 L four-necked flask with jacket and stirred mechanically (paddle agitator), heated to 27° C. for reaction;
2) The pH was stabilized at 6.3 by the addition of a 20% sodium carbonate solution;
3) The sample was taken for HPLC analysis at 15-hour, 23-hour, 39-hour, 47-hour and 63-hour reaction period.

The control data for the samples are presented in Table 22.

TABLE 22

| Reaction time(h) | Compound 1 | Compound 2 | Compound 2-A |
|---|---|---|---|
| 15 | 35.2% | 55.0% | 7.1% |
| | 33.1% | 56.6% | 7.9% |
| 23 | 18.7% | 71.4% | 7.6% |
| | 19.3% | 69.9% | 8.4% |
| 39 | 6.6% | 77.1% | 13.4% |
| | 6.2% | 76.9% | 14.1% |
| 47 | 4.1% | 81.6% | 11.8% |
| | 4.3% | 78.2% | 14.4% |
| 63 | 0.4% | 79.7% | 15.1% |
| | 0.5% | 72.1% | 22.3% |

The results showed that the reaction process was smooth and basically complete when enzyme supernatant was used to replace the cell crushing fluid. Moreover, since the enzyme supernatant contains PBS buffer, not adding buffer here will have no negative impact on the reaction.

### Example 13. Preliminary Scale-Up Enzymatic Reaction Test

TABLE 23

| Reagent | Amount | |
|---|---|---|
| Compound 1 | 40.0 | g |
| Toluene | 120.0 | mL |
| IRED cell | 40.0 | g |
| NADP | 280.0 | mg |
| GDH | 20 | mL |
| glucose | 64.5 | g |
| 0.1M PBS buffer solution | 95.5 | mL |

### Procedure:

1) The materials (concentration of substrate: about 100 g/L) were added into a 500 mL three-necked flask with jacket and stirred mechanically, heated to 27° C. for reaction;
2) The pH was stabilized at 6.3 by the addition of a 20% sodium carbonate solution;
3) The sample was taken for HPLC analysis after a 22-hour, 40-hour, 45-hour reaction periods;
4) store in the freezer.

The data for the samples are presented in Table 24.

TABLE 24

| Reaction time (h) | Compound 1 | Compound 2 | Compound 1 |
|---|---|---|---|
| 22 | 11.0% | 78.2% | 8.5% |
| | 11.1% | 77.6% | 9.2% |
| 40 | 1.8% | 73.6% | 19.9% |
| | 1.6% | 79.1% | 15.7% |
| 45 | 0.9% | 81.3% | 14.5% |
| | 0.9% | 83.7% | 12.7% |

The preliminary scale-up experiment shows that when the cell and substrate feeds are both 100 g/L, only 0.9% of the substrate raw material is left within 45 hours, and the reaction is basically complete.

### Example 14: Post-Treatment Procedures of Enzymatic Reaction for Preparation of TL-010-2 Free Base

### Procedure One:

20 g of Compound 1 was used as the substrate and reacted according to the method of Example 13 for 45 hours to obtain the enzymatic reaction solution. The solution was directly centrifuged, and the supernatant was concentrated by reduced pressure at 50° C. until the solvent was reduced to approximately 40 mL (2 v/w). Methanol (200 mL, 10 v/w) was added and the evaporation was carried out twice, each time leaving about 40 mL (2 v/w) of solvent. After stirring for 15 hours, the mixture was filtered. The cellular debris was centrifuged, and 200 mL (10 v/w) of methanol was added to make a paste, followed by filtration. The methanol phases were combined. To the methanol phase, 85% hydrazine hydrate (0.21 mol, 3.0 eq) was added and stirred at an external temperature of 80° C. for 1 hour. After cooling in an ice bath, a small amount of solid precipitated, which was filtered out. The mother liquor was concentrated by reduced pressure at 40° C. to remove most of the solvents (leaving about 60 mL, 3 v/w). Then, 200 mL (10 v/w) V of DCM was added and the system was concentrated by reduced pressure to about 60 mL (3 v/w). An additional 400 mL (20 v/w) of DCM was added, and the mixture was stirred overnight at room temperature to form a paste. The yield of TL-010-2, as determined by HPLC, was 89.3%. Procedure Two:

40 g of Compound 1 was used as the substrate and reacted according to the method of Example 13 for 45 hours to obtain the enzymatic reaction solution. The solution was concentrated by reduced pressure at 50° C. to remove most of the solvents and water (leaving about 80 mL, 2 v/w). It was then dried with DCM three times and with methanol once (each drying left about 80 mL (2 v/w) of solvent in the system). After making a paste with 400 mL (10 v/w) of methanol for 15 hours, the mixture was filtered, and the methanol phase was collected. To the methanol phase, 85% hydrazine hydrate (0.42 mol, 3.0 eq) was added and stirred at an external temperature of 80° C. for 1 hour. After cooling in an ice bath, a small amount of solid precipitated, which was filtered out. The mother liquor was concentrated by reduced pressure at 40° C. to remove most of the solvents (leaving about 120 mL, 3 v/w). Then, 400 mL (10 v/w) of DCM was added and the system was concentrated by reduced pressure to about 120 mL (3 v/w). An additional 800 mL (20 v/w) of DCM was added, and the mixture was stirred overnight at room temperature to form a paste. The yield of TL-010-2, determined by HPLC, was 85.2%.

### Procedure Three:

20 g of Compound 1 was used as the substrate and reacted according to the method of Example 13 for 45 hours to obtain the enzymatic reaction solution. The solution was directly centrifuged, and a small amount of solid floated between the toluene and water layers, which was then centrifuged again to collect the cellular pellet and mother liquor. The mother liquor was concentrated by reduced pressure at 50° C. to remove toluene and water, then made into a paste with methanol, filtered, and the methanol phase was collected. To the methanol phase, 85% hydrazine hydrate (0.21 mol, 3.0 eq) was added and stirred at an external temperature of 80° C. for 1 hour. After cooling in an ice bath, a small amount of solid precipitated, which was filtered out. The mother liquor was concentrated by reduced pressure at 40° C. to remove most of the solvents (leaving about 60 mL, 3 v/w V). Then, 200 mL (10 v/w V) of DCM was added and the system was concentrated by reduced pressure to about 60 mL (3 v/w). An additional 400 mL (20 v/w V) of DCM was added, and the mixture was stirred overnight at room temperature to form a paste. The yield of TL-010-2, determined by HPLC, was 44.64%.

The cake was made into a paste with methanol (200 mL, 10 v/w), filtered, and the methanol phase was collected. The same procedure with 85% hydrazine hydrate was followed, and the yield of TL-010-2, determined by HPLC, was 44.64%. The overall yield of the mother liquor and the cake combined was 89.28%.

### Example 15: Kilogram-Level Production of Chiral Amine Intermediates

### Step 1: Enzymatic Synthesis of Compounds 2 and 2A and Product Post-Treatment

1. In a 50 L multi-port reaction kettle, compound 1 (2.32 Kg, 7.99 mol, 1 eq), glucose (3.744 Kg, 20.78 mol, 2.6 eq), NADP (8.12 g, 0.0103 mol, 0.00074 eq), toluene (3 v/w), GDH (solution, 0.5 v/w), and IRED enzyme supernatant (4 v/w) were added, with a total volume of 23.5 L. The mixture was stirred mechanically and reacted at 29° C.
2. A 10% solution of sodium carbonate was added dropwise to adjust the pH value of the reaction solution to a stable level of 6.3.
3. After 42 hours of reaction, samples were taken and in-process HPLC analysis was performed. For sample preparation, 0.3 mL of the reaction liquid and 2.0 mL of DMSO were added to a 3 mL centrifuge tube, which resulted in slight heat release. The supernatant was taken after centrifugation. An injection of 0.3 μL was made at 210 nm. The HPLC IPC reaction completion specification was set at ≤2.0% compound 1 remaining.
4. After the reaction was completed, the enzyme reaction solution is post-treated and the methanol phases were combined according to the method of Procedure One of Example 14 and used directly in the next step of the reaction.

### Step 2: TL-010-2 Synthesis

1. To the combined methanol phases from Step 1, 85% hydrazine hydrate (1.411 Kg, 23.97 mol, 3 eq) was added. The mixture was heated to 80° C. and stirred the reaction for 1 hour.
2. An IPC sample was taken to check for reaction completion. An injection of 0.5 μL was made, 210 nm. The HPLC IPC reaction completion specification is <0.5% compound 2/2-A remaining.
3. Heating was stopped, and the mixture was allowed to cool down naturally while stirring overnight.
4. The mixture was distilled under reduced pressure (with a temperature of 40° C.) to evaporate most of the solvent, leaving a remaining volume of about 3.0 v/w. Dichloromethane (DCM) was used to azeotrope the mixture twice, adding 5.0 v/w each time, maintaining a remaining volume of 3.0 v/w each time. Then, 20.0 v/w of DCM was added and beat at room temperature (15° C.) overnight.
5. The system was cooled to 6° C., followed by filtering the mixture. The resulting filter cake was then rinsed with cold dichloromethane (DCM) at 5° C., using a volume-to-weight ratio of 2.0 v/w.
6. The mother liquor from Step 5 was concentrated to a residue and isolated as Product 1.
7. DCM (3.0 v/w) was added to the filter cake from Step 5 and agitated at room temperature (15° C.) overnight.
8. The mixture was cooled to 6° C., filtered, and the filter cake was rinsed with cold DCM (5° C., 2.0 v/w).
9. The mother liquor was concentrated to a residue and isolated as Product 2.
10. The combined mother liquor Product 1 and Product 2 were the 1st batch of product as an oil. The cake was washed with DCM (2 v/w) and dried to afford a 2nd crop of product as a solid.
11. The first batch (an oil) was 0.962 Kg (5.929 mol, 74.2%) and the second batch (a solid) was 0.1212 Kg (0.747 mol, 9.3%).

Total combined yield was 1.0832 Kg (6.677 mol, 83.6%).

Corrected for purity of TL-010-02 free base, the first crop was 90.6% pure, 0.8716 Kg (5.373 mol, 67.2%) and the second crop was 99.1% pure, 0.1201 Kg (0.740 mol, 9.3%) and the combined total was 0.9917 Kg (6.113 mol, 76.4%).

### Step 3: TL-010-2 DiHCl Salt Synthesis

1. TL-010-2 (1 eq) and EtOH (10.0 v/w) were added to a three-mouth reaction flask, and mechanically stirred at room temperature.
2. After dissolving, 10% HCl/EtOH solution (4.401 Kg, 27.74 mol, 5 eq) was added dropwise at controlled temperature (10±5° C.), and solids were precipitated during the dropwise addition process, and the temperature was controlled below 30° C.
3. After adding, set the temperature at 80° C. and reflux stir for 0.5 h; naturally cool to room temperature, stir at room temperature for 12 h.
4. The system was cooled to 5±3° C. in 2 h, stirred for 1 h, filtered quickly under nitrogen protection, and the filter cake was washed with 2.0 v/w cold ethanol (5±3° C.), and the wet product was dried at 50° C. under reduced pressure (water content<1.0%) to obtain the product, which was light yellow or white in color. Weigh the mother liquor and test the product content to calculate the loss of the product in the mother liquor.
5. The combined overall yield for two batches comprising the isolated main product as well as the mother liquors and correcting for purity is as follows:
Total amount TL-010-2 DiHCl is 1.133 Kg (98.3% purity, ee purity>98%, 78% yield).

### Characterization of Minor (1S)-Enantiomer Impurity

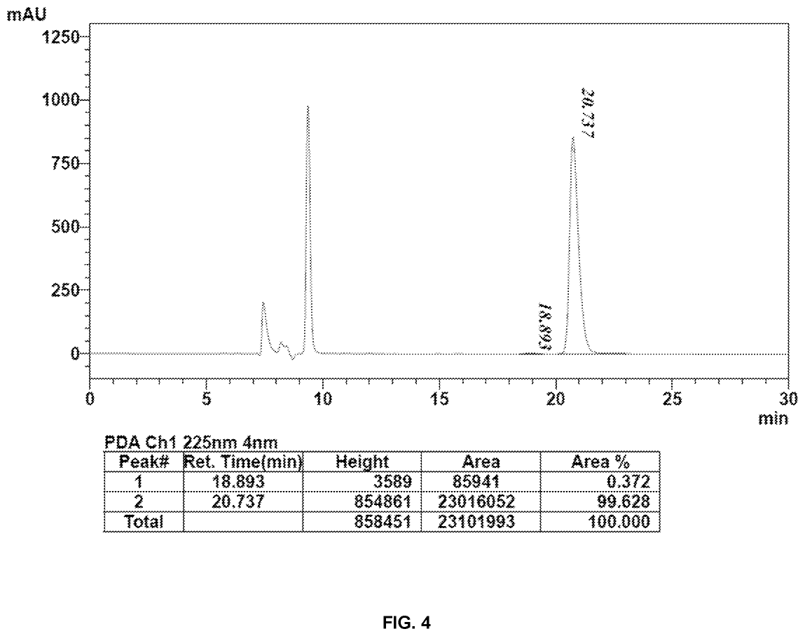

In the chiral HPLC chromatogram of TL-010-2 dihydrochloride salt (FIG. 4), two well-resolved peaks are observed under the chiral HPLC conditions described in the present disclosure. The major peak (Peak 2) elutes at a retention time of about 20.737 minutes with an area percentage of 99.628%, corresponding to the desired (1R)-enantiomer of TL-010-2 dihydrochloride. A minor peak (Peak 1) elutes at a retention time of about 18.893 minutes with an area percentage of about 0.372%. Based on the fact that TL-010-2 contains only a single chiral center and that the chiral HPLC chromatogram shows exactly two well-resolved enantiomeric peaks, this minor peak is reasonably assigned to the (1S)-enantiomer impurity formed as an inherent minor by-product of the asymmetric reduction pathway.

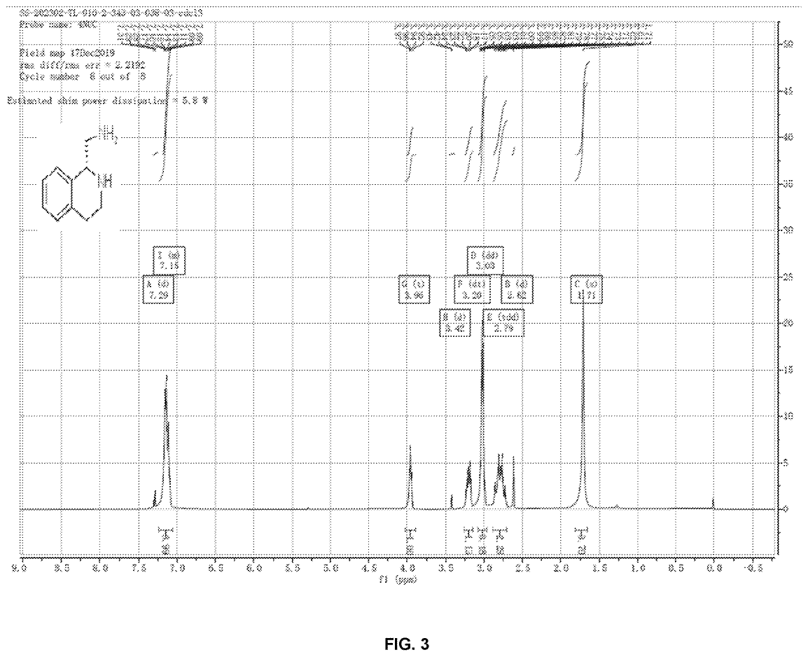

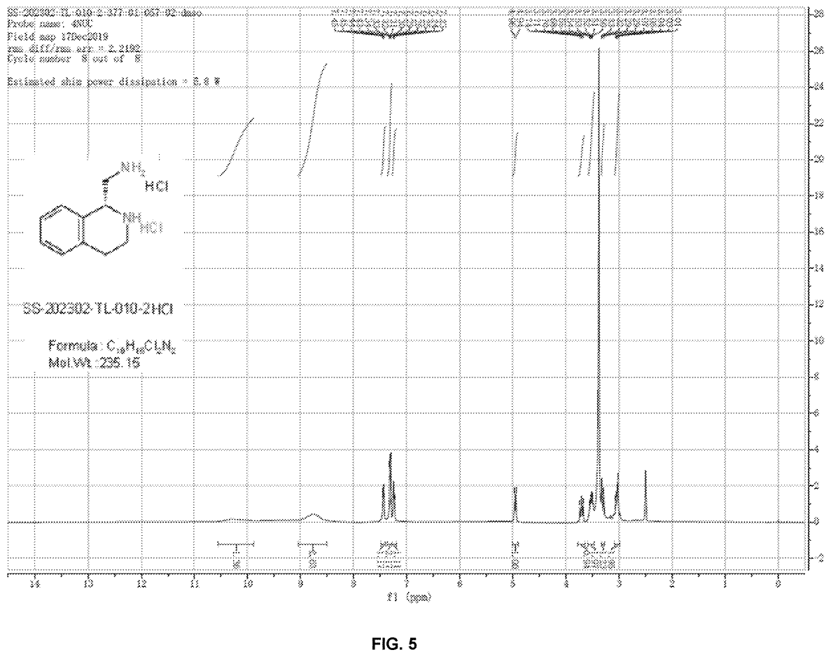

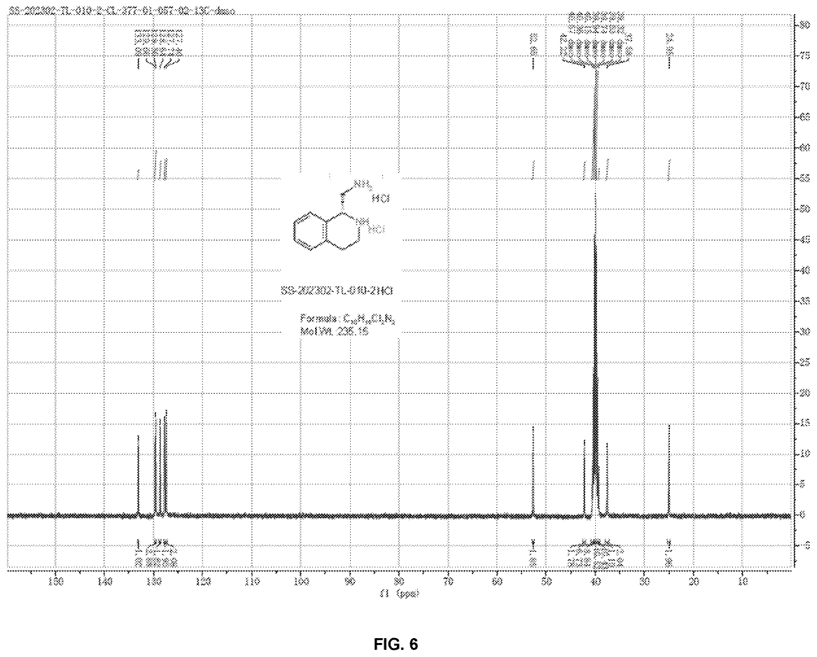

The free base of TL-010-2 and its dihydrochloride salt have been characterized by ¹H-NMR and ¹³C-NMR spectroscopy (FIGS. 3, 5 and 6), and the spectral data are consistent with the proposed structures. Since the (1S)-enantiomer impurity has the same constitution and molecular formula as TL-010-2 except for the opposite configuration at the single chiral center, a person skilled in the art would expect its NMR and mass spectral features to be substantially the same as those of TL-010-2, with only minor differences attributable to the enantiomeric relationship.

Accordingly, the minor peak at a retention time of about 18.893 minutes serves as a marker peak for the (1S)-enantiomer impurity of TL-010-2 dihydrochloride, which is an inherent minor by-product of the enzyme-catalyzed asymmetric reduction pathway and a process-indicative marker impurity characteristic of the present manufacturing process. In preferred embodiments, the content of this impurity is controlled to not more than 0.5% by area percentage in the chiral HPLC chromatogram.

### Example 16, Preliminary Stability Experiment of TL-010-2 Free Base and TL-010-02 Dihydrochloride Salt

Stability tests were conducted on the obtained TL-010-2 free base and TL-010-2 DiHCl, see Table 25. The TL-010-2 free base exhibited a decrease in purity to approximately 90% after storage at 5±3° C. for 60 days. Furthermore, when stored at 25±2° C. and 60±5% RH for 60 days, the purity declined to around 65.38%. Conversely, the TL-010-2 DiHCl was stored at 5=3° C. for 60 days and under conditions of 25±2° C. and 60±5% relative humidity (RH) for 60 days, with purity levels remaining above 99% in both scenarios. Therefore, for long-term storage, TL-010-2 DiHCl is recommended due to its superior stability and purity retention.

TABLE 25

| | | Purity % at different storage time (day) | | | | | |
|---|---|---|---|---|---|---|---|
| Product | Storage condition | 0 | 14 | 21 | 28 | 36 | 60 |
| TL-010-2 | 5 ± 3° C. (Sealed stored in | 96.90 | 96.87 | 96.87 | 96.45 | 96.07 | 89.97 |
| free base | refrigerator without humidity | | | | | | |
| (purity, %) | controlled) | | | | | | |
| | 25 ± 2° C. (Sealed stored in | 96.90 | 90.91 | 88.21 | 85.24 | 82.96 | 65.38 |
| | stability chamber with humidity | | | | | | |
| | controlled) | | | | | | |
| TL-010-2 | 5 ± 3° C. (Sealed stored in | 99.82 | 99.83 | 99.84 | 99.84 | 99.80 | 98.86 |
| DiHCl | refrigerator without humidity | | | | | | |
| (purity, %) | controlled) | | | | | | |
| | 25 ± 2° C. (Sealed stored in | 99.82 | 99.82 | 99.72 | 99.84 | 99.74 | 99.85 |
| | stability chamber with humidity | | | | | | |
| | controlled) | | | | | | |

The present application combines bio-enzymatic catalysis with chemical methods, offering the advantages of high yield and high purity, making it more suitable for large-scale industrial production. Compared to existing traditional chemical methods, the present application features a simplified enzymatic process, operates under mild reaction conditions, and is simple and convenient to control. Moreover, it can reduce the use of energy and organic solvents, achieving green production. This method helps to address the unresolved industrial challenge of high-purity (R)-prazinquantel separation and purification, paving the way for preclinical and clinical pharmacological evaluation of (R)-prazinquantel and the large-scale industrial production of (R)-prazinquantel to enter the international market.

The features, structures, effects, and the like described in the above-described embodiments include at least one embodiment of the present disclosure, but the present disclosure is not limited only to one embodiment. Further, the features, structures, effects, and the like illustrated in each embodiment may be combined or modified to other embodiments by those skilled in the art. Therefore, contents related to the combination or the modification should be interpreted to be included in the scope of the disclosure.

In addition, while the present disclosure has been particularly described with reference to exemplary embodiments, the present disclosure is not limited thereto. It will be understood by those skilled in the art that various modifications and applications, which are not illustrated in the above, may be made without departing from the spirit and scope of the present disclosure. For example, each component illustrated in the embodiments may be modified and made. It should be interpreted that differences related to these modifications and applications are included in the scope of the disclosure defined in the appended claims.

We claim:

1. A method for preparing a chiral amine compound and its salt comprising the following steps:
(1) causing compound 1 to react in the presence of imine reductase, NADP, glucose dehydrogenase, glucose, a solvent, and a co-solvent to obtain compound 2 and/or compound 2-A;
(2) converting the compound 2 and/or compound 2-A into the chiral amine compound (1R)-(−)-1-aminomethyl-1,2,3,4-tetrahydroisoquinoline (TL-010-2);
wherein, the structural formula of the compound 1 is: 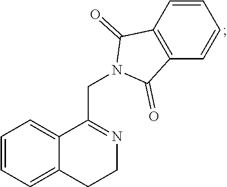 [SMILES: O=C1c2ccccc2C(=O)N1CC1=NCCc2ccccc21]
the structural formula of the compound 2 is: 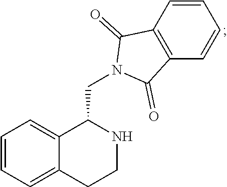 [SMILES: O=C1c2ccccc2C(=O)N1C[C@@H]1NCCc2ccccc21]
the structural formula of the compound 2-A is: 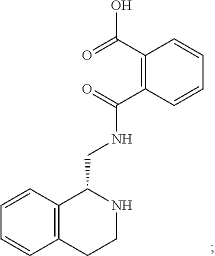 [SMILES: O=C(O)c1ccccc1C(=O)NC[C@@H]1NCCc2ccccc21]
and the structural formula of the TL-010-2 is

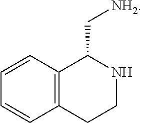 [SMILES: NC[C@@H]1NCCc2ccccc21]

2. The method of claim 1, wherein an initial concentration of the compound 1 is 30 to 100 g/L.

3. The method of claim 2, wherein the initial concentration of the compound 1 is 50 to 100 g/L.

4. The method of claim 1, wherein in step (1), a pH is maintained in the range of 5 to 7; or,
in step (1), a reaction temperature is controlled to be 25° C. to 35° C.

5. The method of claim 4, wherein in step (1), the pH is maintained in the range of 6 to 6.5; or,
in step (1), the reaction temperature is controlled to be 25° C. to 30° C.

6. The method of claim 1, wherein the solvent is PBS buffer solution or water; and/or,
the co-solvent is one or more of ethyl acetate, isopropyl alcohol, dimethyl sulfoxide, toluene, isopropyl ether, methyl tert-butyl ether, and methanol.

7. The method of claim 1, wherein the co-solvent is toluene.

8. The method of claim 1, wherein an amount of NADP is 0.07 wt % to 1.5 wt % of an amount of compound 1.

9. The method of claim 8, wherein the amount of NADP is 0.1 wt % to 1.5 wt % of the amount of compound 1.

10. The method of claim 1, wherein a molar amount of glucose is 1 to 3 times a molar amount of compound 1; and/or
an amount of glucose dehydrogenase is 25 wt % to 75 wt % of an amount of compound 1.

11. The method of claim 10, wherein a molar amount of glucose is 2 to 3 times a molar amount of compound 1.

12. The method of claim 10, wherein an amount of glucose dehydrogenase is 40 wt % to 60 wt % of an amount of compound 1; and/or
a concentration of a crude enzyme solution of glucose dehydrogenase is 50 g/L in a reaction system of step (1).

13. The method of claim 1, wherein the imine reductase is from Escherichia coli or Pichia pastoris, and fed in a form of enzyme powder, whole cells, cell disrupted fluid, or supernatant obtained from cell disrupted fluid after separation;
when the imine reductase is fed in the form of enzyme powder, a feeding quality of the enzyme powder of the imine reductase is 0.3 to 5 times a feeding quality of the compound 1; or 0.5 to 5 times a feeding quality of the compound 1;
when the imine reductase is fed in the form of whole cells or cell disrupted fluid, a feeding quality of the whole cells or a feeding quality of the cells in the cell disrupted fluid is 1 to 10 times a feeding quality of the compound 1;
When the imine reductase is fed in the form of the supernatant obtained from the cell disrupted fluid, a feeding amount of the supernatant is 4 to 10 mL of the supernatant for every 1 g of the compound 1.

14. The method of claim 1, wherein after the completion of step (1), a mixture is subjected to centrifugation or vacuum distillation, DCM and/or methanol is added successively with stirring, and then filtered to collect an organic phase and a filter cake; a step of adding DCM and/or methanol to the filter cake, carrying out slurrying and filtration, and collecting an organic phase, which is performed once or multiple times; and the organic phases are combined and directly used for the reaction in step (2).

15. The method of claim 1, wherein the compound 2 and/or compound 2-A react in the presence of hydrazine hydrate and methanol to obtain the TL-010-2.

16. The method of claim 1, wherein the salts of the chiral amine compound comprise hydrochloride, hydrobromide, oxalate, acetate, lactate, phosphate, maleate, tartrate, citrate, gluconate, sulfate, mesylate, or other sulfonate salts.

17. The method of claim 1, wherein the method further includes a step of converting the TL-010-2 into (1R)-(−)-1-aminomethyl-1,2,3,4-tetrahydroisoquinoline dihydrochloride salt (TL-010-2 dihydrochloride salt) in the presence of hydrochloric acid in ethanol and methanol, wherein the structural formula of TL-010-2 dihydrochloride salt is: 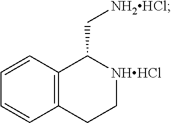
or
the method further includes a step of converting the TL-010-2 into (1R)-(−)-1-aminomethyl-1,2,3,4-tetrahydroisoquinoline dihydrobromide salt (TL-010-2 dihydrobromide salt) in the presence of hydrobromic acid and methanol, wherein the structural formula of TL-010-2 dihydrobromide salt is 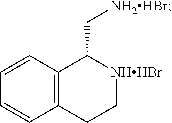
or
the method further includes a step of converting the TL-010-2 into (1R)-(−)-1-aminomethyl-1,2,3,4-tetrahydroisoquinoline oxalate salt (TL-010-2 oxalate salt) in the presence of oxalic acid and methanol, wherein the structural formula of TL-010-2 oxalate salt is 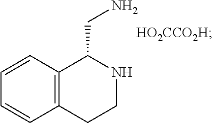 [SMILES: NC[C@@H]1NCCc2ccccc21]
or
the method further includes a step of converting the TL-010-2 into (1R)-(−)-1-aminomethyl-1,2,3,4-tetrahydroisoquinoline maleic salt (TL-010-2 maleic salt) in the presence of maleic acid and methanol, wherein the structural formula of TL-010-2 maleic acid salt is

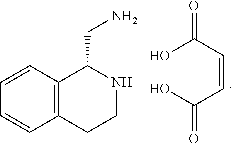 [SMILES: NC[C@@H]1NCCc2ccccc21 ; O=C(O)/C=C\C(=O)O]

18. A salt of a chiral amine having a structure of Formula

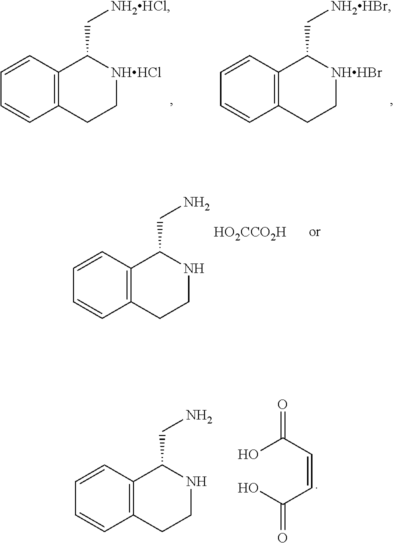

19. A method for preparing (R)-praziquantel comprising the following steps:
1) causing compound 1 to react in the presence of imine reductase, NADP, glucose dehydrogenase, glucose, a solvent, and a co-solvent to obtain compound 2 and/or compound 2-A;
2) converting the compound 2 and/or compound 2-A into a chiral amine compound TL-010-2; or further converting the chiral amine compound TL-010-2 into a salt of TL-010-2;
3) Preparing the (R)-praziquantel using the chiral amine compound TL-010-2 or the salt of TL-010-2 thereof as the starting material;
wherein, the structural formula of the compound 1 is: 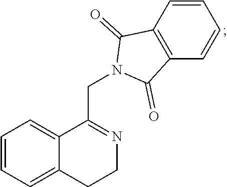 [SMILES: O=C1c2ccccc2C(=O)N1CC1=NCCc2ccccc21]
the structural formula of the compound 2 is: 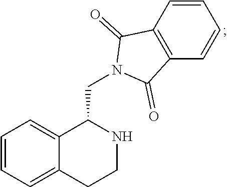 [SMILES: O=C1c2ccccc2C(=O)N1C[C@@H]1NCCc2ccccc21]
the structural formula of the compound 2-A is: 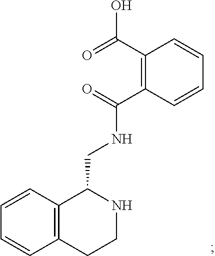 [SMILES: O=C(O)c1ccccc1C(=O)NC[C@@H]1NCCc2ccccc21]
the structural formula of the chiral amine compound TL-010-2 is 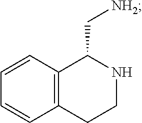 [SMILES: NC[C@@H]1NCCc2ccccc21]
the structural formula of the salts of TL-010-2 are:

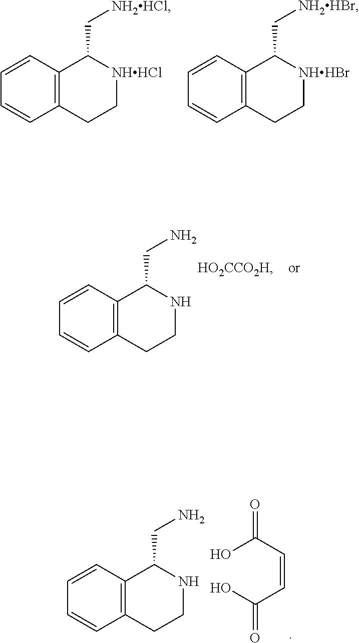

20. The salt of the chiral amine of claim 18, wherein the salt is TL-010-2 dihydrochloride salt with an enantiomeric excess (ee) of >98%, and which comprises less than 2%, preferably less than 0.5% of(S)-isomer impurity as a marker for reaction pathway, as determined by chiral HPLC analysis, wherein the structure of the TL-010-2 dihydrochloride salt is 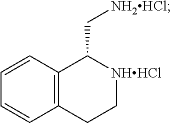
and/or
a TL-010-2 dihydrochloride salt having a chemical purity of remaining above 99% after storage for 60 days at 25±2° C. and 60±5% relative humidity in a sealed container; wherein the structure of the TL-010-2 dihydrochloride salt is [chemical structure]
